(19) United States
(12) Patent Application Publication
(10) Pub. No.: US 2023/0076434 A1
(43) Pub. Date: Mar. 9, 2023

(54) A METHOD FOR ISOLATING A MICROORGANISM

(71) Applicant: TECHNION RESEARCH & DEVELOPMENT FOUNDATION LIMITED, Haifa (IL)

(72) Inventors: Naama GEVA-ZATORSKY, Nahalal (IL); Tal GEFEN, Karmiel (IL)

(21) Appl. No.: 17/796,769

(22) PCT Filed: Feb. 3, 2021
(86) PCT No.: PCT/IL2021/050128

§ 371 Date: Aug. 1, 2022

Related U.S. Application Data
(60) Provisional application No. 62/969,197, filed on Feb. 3, 2020.

(51) Int. Cl. G01N 33/569; C12Q 1/689; C12Q 1/04

(52) U.S. Cl. CPC G01N 33/56911; C12Q 1/689; C12Q 1/04; G01N 2469/10

(57) ABSTRACT

The present invention, in some embodiments thereof, is directed to a method for isolating a target microorganism from a sample, including contacting a sample with an antibody having specific affinity to the target microorganism, wherein the antibody is produced by immunizing a host organism using a selected target microorganism, wherein the selected target microorganism is selected by contacting the sample with one or more polynucleotide molecules each having specific affinity to one target microorganism, and determining the presence of the target microorganism in the sample.

### CROSS-REFERENCE TO RELATED APPLICATIONS

This application claims the benefit of priority of U.S. Provisional Patent Application No. 62/969,197 titled: “METHOD FOR ISOLATING A MICROORGANISM”, filed Feb. 3, 2020, the contents of which are incorporated herein by reference in their entirety.

### FIELD OF INVENTION

The present invention, in some embodiments thereof, is in the field of microbiology, microbiome, and related applications.

### BACKGROUND

The microbiota has been recently shown to have major effects on human health. The composition of the microbiota changes in health and diseases states. Studies have shown that the microbiota composition can have beneficial effects on patients. These include both effects on the health state and on response to therapy. Recently, the microbiota as a whole, has been implicated to clinical practice, in forms of fecal transplantations (FMT). The first clinical indication, and the most successful one to date, was FMT, to treat Clostridium difficile diarrhea. However, currently, there are many clinical trials of FMT treatment for a variety of indication. Important to note that the common fecal source is a healthy human donor. One major challenge in FMT treatment is the microbiota composition of the transplanted feces. In some cases, even traces of unwanted microbes can disqualify the fecal transplantation. A method for eliminating unwanted microbes from a fecal composition, so as to requalify feces for transplantation (e.g., by FMT) in patients, is greatly needed.

### SUMMARY

According to a first aspect, there is provided a method for isolating a target microorganism from a sample comprising a plurality of microorganisms, the method comprising contacting the sample with an antibody having specific affinity to the target microorganism, wherein the antibody being produced by immunizing a host organism using a selected target microorganism, wherein the selected target microorganism is selected by contacting a fraction of the sample with one or more polynucleotide molecules each having specific affinity to one target microorganism, and determining the presence of the target microorganism in the fraction of the sample, thereby isolating a target microorganism from the sample comprising a plurality of microorganisms.

According to another aspect, there is provided a method for isolating a target microorganism from a sample, the method comprising the steps of: (a) providing a fraction of the sample comprising a plurality of microorganisms; (b) contacting the fraction of the sample with one or more polynucleotide molecules each having specific affinity to one target microorganism, and determining the presence of the one or more target microorganism in the fraction of the sample; (c) selecting the one or more target microorganisms determined to be present in the fraction of the sample and immunizing a host organism using one of the selected target microorganisms, thereby producing an antibody having specific affinity to the one selected target microorganism; and (d) contacting the sample with the produced antibody having specific affinity to the one selected target microorganism, thereby isolating a target microorganism from the sample.

According to another aspect there is provided a sample obtained by the herein disclosed method.

According to another aspect, there is provided a composition comprising the herein disclosed sample and a pharmaceutically acceptable carrier.

In some embodiments, the microorganism is selected from the group consisting of: bacteria, fungi, archaea, protozoa, and algae.

In some embodiments, the selected microorganism is a specific species or a specific strain of a microorganism.

In some embodiments, the microorganism is a pathogenic microorganism.

In some embodiments, the microorganism is a probiotic microorganism.

In some embodiments, contacting comprises contacting the sample with a plurality of polynucleotide molecules each having specific affinity to one target microorganism.

In some embodiments, the sample is selected from the group consisting of: a sample derived from a subject, a soil sample, and a water sample.

In some embodiments, the sample derived from a subject is a stool sample of the subject.

In some embodiments, the method further comprises a step of enriching the sample with the isolated microorganism.

In some embodiments, the method further comprises a step of depleting the isolated microorganism from the sample.

In some embodiments, the method further comprises a step of modulating a sample such that the amount, distribution, abundance, or any combination thereof, is modified so as to be compatible for administering to a subject in need thereof.

In some embodiments, modulating comprises increasing, elevating, or any equivalent thereof.

In some embodiments, modulating comprises decreasing, reducing, or any equivalent thereof.

In some embodiments, the modulating is tailored to the physical state of the subject.

In some embodiments, the method further comprises a step of determining the condition of the subject prior to administration.

In one embodiment, wherein a subject is determined to comprise a pathogenic microorganism, the method comprises depleting the pathogenic microorganism from a sample or a fraction thereof, prior to administration to the subject.

In one embodiment, wherein a subject is determined to lack a probiotic microorganism, the method comprises enriching a sample or a fraction thereof with the probiotic microorganism, prior to administration to the subject.

In some embodiments, the sample or fraction thereof is an autologous sample.

In some embodiments, the sample or fraction thereof is an allogeneic sample.

In some embodiments, the method further comprises a step of determining the produced antibody has increased specific affinity to the one selected target microorganism compared to control.

In some embodiments, the composition is for use in treatment of a subject afflicted with a disease.

In some embodiments, the disease is selected from the group consisting of: Clostridium difficile diarrhea, inflammatory bowel disease, irritable bowel disease, cancer, and diabetes.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 includes an illustration of a non-limiting scheme of fluorescent in situ hybridization (FISH). The probe binds to a target molecule (e.g., DNA). The target molecule (e.g., DNA) is denatured to enable hybridization. Then, the probes hybridizes, and subsequently analysis is performed based on the dye linked to the probe (e.g., a fluorescent moiety, etc.).

FIG. 2 includes an illustration of a non-limiting scheme of the method of the invention. The method comprises designing a specific DNA fluorescent probe(s) that specifically binds to a bacteria of interest. Sorting out these bacteria by fluorescent in situ hybridization (“FISHing”) with the probe, is followed by immunizing a host animal in order to generate antibodies. A host animal can be, but is not limited to a chicken, In the case of a chicken, the antibody can be collected from the egg yolk in high amounts, and the chicken can be kept alive, while continuing to lay eggs with large quantities of the antibody. Further, the chicken immunoglobulin Y (IgY) antibody is inert to mammalian hosts and to the bacteria, while bacteria can bind to protein A/G on other antibodies. Nonetheless, the antibody can be produced in a mammalian host, e.g., a mice, or a rabbit. The method of the invention can be done for combinations of a plurality of different bacteria at once.

FIGS. 3A-3E include graphs showing flow cytometry of a probe hybridizing to a bacteria of interest: Clostridium perfringens (3A); Bacteroides fragilis (3B); Parabacteroides johnsonii (3C); Blautia coccoides (3D); and Escherichia nissle (3E). FISH-DNA probes were designed according the bacteria of interest. Probes were hybridized with the bacteria in vitro, and fluorescence-activated cell sorting (FACS) followed thereafter.

FIGS. 4A-4G include illustrations of non-limiting schemes depicting the method of the invention and graphs. (4A) describes a process for production of specific antibodies to target bacteria of interest from natural sources, such as, but not limited to stool. These antibodies can be used, for example, to enrich for the target bacteria or to deplete the target bacteria from the natural source. The process comprises immunizing a host, e.g., a chicken, a rabbit, a mice, etc., in order to produce antibodies against the bacteria of interest. (4B-4C) are graphs of flow cytometry analysis showing the specificity of the antibody. (4B) shows bacterial events as detected by flow cytometry, for physical parameters. Size— FSC-A and granularity SSC-A, each dot represents single bacteria. (4C) Background control of signal intensity (R-670/30-A) by omitting the specific primary antibody from the assay. (4D) B. fragilis was incubated with specific primary antibodies followed by secondary antibody and tested by flow cytometry. Specific detection intensity was found to be at 4 log above the background. The antibody was further shown to specifically bind only to a specific species, with the highest affinity to the strain to which it was raised against, and no affinity to another species (4E, left most bar). (4F-4G) are illustrations of non-limiting examples of two methods for isolating a bacteria of interest by, e.g., antibodies (identification and targeting can also be performed with a FISH probe), using FACS (4F), or magnetic beads separation (4G).

FIGS. 5A-5E include graphs showing specific bacterial isolation using beads. Flow cytometry of analysis using an antibody binding to the bacteria, with magnetic beads separation is shown. B. fragilis or non-relevant bacteria were stained with Hoechst DNA labeling. Following three washing steps, bacteria were incubated with specific antibodies coated beads. (5A) Detection of magnetic beads (gated) physical parameters. Size-FSC-A, granularity— SSC-A. Single bacteria events can be detected on the lower left corner of the graph; (5B) and (5C) Physical parameters detection are presented in log scale to enable higher resolution of bacterial events together with magnetic beads detection; (5D) Gated beads intensity following incubation with stained non-relevant control bacteria; and (5E) is gated beads with following incubation with stained B. fragilis. Intensity of the specific beads was found to be at least two fold above the negative control. The same procedure can be done for combinations of bacteria at once.

FIGS. 6A-6C include graphs showing general detection of bacteria by flow cytometry. (6A) Detection of bacteria (gated) physical parameters. Size— FSC-A, granularity— SSC-A. Single bacteria is detected in the gated events; (6B) and (6C).

FIGS. 7A-7D include graphs showing specific detection of B. fragilis using specific IgY antibodies. Binding of IgY to the bacteria is detected by a secondary antibody—Rabbit anti-chicken Alexa Fluor 647 conjugate. (7A) 93.6% of right gated B. fragilis. Left gated events are consider as debris. (7B) No binding to Bifidobacterium adolescentis. Right gate. (7C) Minimal binding of B. longum with low fluorescence intensity in comparison to specific B. fragilis detection (7A). (7D) No binding to B. thetaiotaomicron. Right gate.

FIGS. 8A-8C include graphs showing staining of a mixture of B. fragilis with other bacteria. (8A) B. fragilis with B. adolescentis. (8B) B. fragilis with B. longum. (8C) B. fragilis with B. thetaiotaomicron. No alteration in fluorescence intensity of specific B. fragilis detection were observed in the mixtures examined.

FIGS. 9A-9C include graphs of sorted B. fragilis and off-target bacteria. (9A) B. fragilis mixed with B. adolescentis, B. longum, and B. thetaiotaomicron, and sorted using FACS AriaIII BD. Specific staining of bacteria carried by IgY B. fragilis specific antibody, and Rabbit anti-chicken Alexa Fluor 647 conjugate. (9B) post sorted, negative bacteria for antibody staining showing less than 2% of stained B. fragilis. Indicating depletion of B. fragilis from bacteria mixture. (9C) post sorted B. fragilis positive sorted, enriched to 89.6% purity.

FIGS. 10A-10I include graphs showing staining of a mixture of B. fragilis with other bacteria. (10A-10E) single stain for B. fragilis, B. vulgatus, Peptostreptococcus magnus, B. adolescentis and Propionibacterium granulosum, respectively. Mixture of stained B. fragilis with: (10F) B. vulgatus; (10G) P. magnus; (10H) B. adolescentis; and (10I) P. granulosum. No alteration in fluorescence intensity of specific B. fragilis detection observed following the mixture.

FIGS. 11A-11C include graphs of sorted B. fragilis and off-target bacteria. (11A)B. fragilis mixed with B. vulgatus, P. magnus, B. adolescentis, and P. granulosum, and sorted using FACS AriaIII BD. Specific staining of bacteria was carried by IgY B. fragilis specific antibody, and Rabbit anti-chicken Alexa Fluor 647 conjugate. (11B) post sorted, negative bacteria for antibody staining showing less than 1% of stained B. fragilis. Indicating depletion of B. fragilis from bacteria mixture. (11C) post sorted B. fragilis positive sorted, enriched to 94.2% purity.

FIGS. 12A-12C include graphs showing physical detection of B. fragilis (12A), human fecal bacteria (12B) and mixture of both (12C). Size— FSC-A, granularity—SSC-A.

FIGS. 13A-13C include graphs for sorting B. fragilis from fecal bacteria mixture. (13A) Labeled B. fragilis with Hoechst 33342 dye (binding DNA within the bacteria). Following labeling, signal was detected in Y axis (Alexa Fluor 405). (13B) Specific antibody staining of Hoechst labeled B. fragilis. 83.7% of double labeled B. fragilis. (13C) Detection of bacteria in human feces prior to the addition of B. fragilis.

FIGS. 14A-14C include graphs for sorted B. fragilis from fecal bacteria mixture. (14A) Pre-sorted mixture of Hoechst labeled (Y axis) and specific antibody labeled (X axis) B. fragilis. (14B) Post-sorted B. fragilis 81.9% purity. (14C) Post-sorting depleted feces from B. fragilis to 93.6% purity.

### DETAILED DESCRIPTION

In some embodiments, a method for isolating a target microorganism from a sample comprising a plurality of microorganisms, is provided.

In some embodiments, the method comprises contacting a sample with an antibody having specific affinity to the target microorganism, wherein the antibody is produced by immunizing or immunization of a host organism using a selected target microorganism, wherein the selected target microorganism is selected by contacting a fraction of the sample with one or more polynucleotide molecules each having specific affinity to one target microorganism, and determining the presence of the target microorganism in the fraction of the sample, thereby isolating a target microorganism from the sample.

In some embodiments, contacting is under conditions sufficient to enable the binding of an antibody to an antigen or an epitope thereof, e.g., such as the antibody was raised against by immunizing a host organism.

In some embodiments, contacting is under conditions sufficient to enable base pairing of the one or more polynucleotides and complementary polynucleotides comprised by the one or more microorganisms of the sample.

In some embodiments, the method comprises a step of providing a sample comprising a plurality of microorganisms. As used herein, the term “providing a sample” comprises obtaining or producing the sample.

In some embodiments, the method comprises a step of contacting a fraction of the sample with one or more polynucleotide molecules each having specific affinity to one target microorganism, and determining the presence of the one or more target microorganism in the sample.

In some embodiments, determining comprises detecting a signal indicative of the hybridization of the one or more polynucleotides having specific affinity to one target microorganism. In some embodiments, hybridization comprises base pairing of the one or more polynucleotides and complementary polynucleotides comprised by the one or more microorganisms of the sample. In some embodiments, the complementary polynucleotides comprised by the one or more microorganisms of the sample comprises DNA and/or RNA polynucleotides.

In some embodiments, the signal indicative of the hybridization comprises any one of: a fluorescent signal, a radioactive signal, and a chromatic signal.

In some embodiments, the one or more polynucleotides having specific affinity to one target microorganism is any one of: fluorescently labeled, radioactively labeled, and chromatically labeled. In some embodiments, the one or more polynucleotides having specific affinity to one target microorganism comprises a molecule or a moiety embedded or incorporated therein. In some embodiments, the molecule or moiety are further recognized and/or bound by a molecule having increased binding affinity to the molecule or moiety, such as a specific antibody (e.g., digoxigenin (DIG) and an anti-DIG antibody) or a binding counterpart (e.g., avidin and biotin). In some embodiments, the antibody or binding counterpart is further linked to an enzyme. In some embodiments, the linked enzyme is capable of catalyzing colorimetric reaction. In some embodiments, the colorimetric reaction comprises a bioluminescent reaction or a chemiluminescent reaction.

In some embodiments, the method comprises a step of selecting the one or more target microorganisms determined to be present in the sample and immunizing a host organism using one of the selected target microorganisms, thereby producing an antibody having specific affinity to the one selected target microorganism.

Methods for immunization of a host organism are common and would be apparent to one of ordinary skill in the art. Non-limiting examples of a host organism suitable for immunization include, but are not limited to, a chicken, a rabbit, and a mouse.

In some embodiments, the method comprises a step of contacting the sample with the produced antibody having specific affinity to the one selected target microorganism, thereby isolating a target microorganism from the sample.

In some embodiments, the method comprises contacting the sample with a plurality of polynucleotide molecules each having specific affinity to one target microorganism.

According to some embodiments, there is provided a method for selecting one or more target microorganisms for immunizing a host organism, the method comprising the steps of: (1) providing a fraction of a sample comprising a plurality of microorganisms; (2) contacting the fraction of the sample with one or more polynucleotide molecules each having specific affinity to one target microorganism, and determining the presence of the one or more target microorganism in the fraction of the sample; and (3) selecting the one or more target microorganisms determined to be present in the fraction of the sample for immunizing a host organism.

In some embodiments, the method further comprises immunizing a host organism using one of the selected target microorganisms determined to be present in the sample or a fraction thereof. In some embodiments, the produced antibody is characterized by having specific affinity to the selected target microorganism.

In some embodiments, the method further comprises a step comprising contacting a sample with the produced antibody. In some embodiments, the method comprises a step of contacting a sample with the produced antibody having specific affinity to the selected target microorganism. In some embodiments, the contacting comprises or results in isolating the target microorganism from the sample or a fraction thereof.

As used herein, the term “plurality” refers to any number or value that is greater than one. In some embodiments, a plurality comprises 2 to 10, 2 to 50, 20 to 100, 2 to 500, 2 to 1,000, or 2 to 10,000. Each possibility represents a separate embodiment of the invention. In some embodiments, a plurality comprises at least 2, at least 5, at least 10, at least 50, at least 100, at least 500, at least 1,000, at least 10,000, or any value and range therebetween. Each possibility represents a separate embodiment of the invention.

In some embodiments, the microorganism is selected from: bacteria, fungi, archaea, protozoa, and algae.

In some embodiments, the bacteria comprises a Bifidobacterium. In some embodiments, Bifidobacterium comprises B. fragilis.

In some embodiments, the targeted microorganism is a pathogenic microorganism. As used herein, the term “pathogenic microorganism” comprises any microorganism which diverts a host organism from homeostasis. In some embodiments, a pathogenic microorganism induces, initiates, promotes, propagates, or any equivalent thereof, of a disease or a symptom associated therewith.

In some embodiments, the targeted microorganism is a probiotic microorganism. As used herein, the term “probiotic microorganism” refers to any microorganism which has or promotes health beneficiary effects on a host organism comprising thereof.

In some embodiments, the selected microorganism is a specific species or a specific strain of a microorganism.

In some embodiments, the sample is selected from: a sample derived from a subject, a soil sample, and a water sample.

In some embodiments, the sample comprises environmental sample. In some embodiments, the method of the invention is directed to isolation of uncultured microorganisms.

In some embodiments, a sample derived from a subject comprises a tissue or a cell of the subject. In some embodiments, a sample derived from a subject comprises bodily fluids of the subject. In some embodiments, a sample derived from a subject comprises a stool sample of the subject.

In some embodiments, the method further comprises a step of enriching a sample with the isolated microorganism.

In some embodiments, the method further comprises a step of depleting the isolated microorganism from the sample. In some embodiments, depleting comprises: removing, eliminating, omitting, or any equivalent thereof, as long as isolated microorganism is not present in the sample. In some embodiments, the sample is devoid of the isolated microorganism upon performing the method of the invention.

In some embodiments, the method, further comprises a step of determining the produced antibody has increased specific affinity to the one selected target microorganism compared to control.

In some embodiments, the method of the invention provides a composition suitable for any application selected from: human-related, marine-related, agriculture, uncultured bacteria, health-relevant bacteria, depletion of bacteria from stool transplantation, and directed bacteria for probiotics.

As used herein, control comprises a microorganism which is not present in the sample before preforming the method of the invention (“pre-treated sample”). In some embodiments, the control is any compound and/or microorganism which does not cross react with the produced antibody. In some embodiments, the control is a microorganism of any different species, line, strain, Glade, phyla, or any equivalent thereof, other than the target microorganism.

As used herein, increased is by at least 5%, at least 10%, at least 25%, at least 50%, at least 100%, at least 250%, at least 500%, at least 750%, at least 1,000%, or any value and range therebetween, compared to control. Each possibility represents a separate embodiment of the invention. In some embodiments, increased is by 5-150%, 10-250%, 5-400%, 10-550%, 50-600%, 100-375%, 250-750%, 300-800%, or 725-1,000%, compared to control. Each possibility represents a separate embodiment of the invention.

Methods for determining antibody specificity are common and would be apparent to one of ordinary skill in the art. Non-limiting examples of such methods, include, but are not limited to, western blot, immunoprecipitation, enzyme-linked immunosorbent assay (ELISA, including direct and indirect ELISA), competitive binding assays, and others.

In some embodiments, the present invention provides a sample obtained by the method of the invention.

In some embodiments, the present invention provides a composition comprising a sample obtained by the method of the invention.

In some embodiments, the composition further comprises an acceptable carrier. In some embodiments, the carrier comprises a pharmaceutically acceptable carrier.

In some embodiments, the composition is for use in treating a subject afflicted with a disease.

In some embodiments, the disease is selected from: Clostridium difficile diarrhea, inflammatory bowel disease (IBD), irritable bowel disease, cancer, and diabetes.

In some embodiments, the composition is suitable for use in cancer immunotherapy.

In some embodiments, the composition is a functional probiotic composition. In some embodiments, the functional probiotics is suitable for use in treating an infant. In some embodiments, the functional probiotic composition comprises Bifidobacterium.

As used herein, the term “cancer” refers to a disease associated or characterized by abnormal cell proliferation. In some embodiments, cancer refers to a disease comprising cell proliferation.

In some embodiments, IBD comprises Crohn's disease, ulcerative colitis, or both.

The terms “sample” and “fraction of a sample” are used herein interchangeably.

In some embodiments, the present invention is directed to a method for producing an antibody, comprising: (a) contacting a sample with one or more polynucleotide molecules each having specific affinity to one target microorganism; (b) selecting a target microorganism determined to be present in the sample; and (c) immunizing a host organism using the selected target microorganism, thereby producing the antibody.

In some embodiments, a composition comprising the produced antibody is provided.

In some embodiments, the produced antibody, a composition comprising thereof, or both, is for use in isolating a microorganism from a sample comprising a plurality of microorganisms.

In some embodiments, the antibody is a chordate derived antibody. In some embodiments, the antibody is a non-mammalian antibody. In some embodiments, the antibody is an avian-derived antibody. In some embodiments, the antibody is a shark derived antibody. In some embodiments, the antibody is a chicken derived antibody. In some embodiments, the antibody is an immunoglobulin Y (IgY) antibody. In some embodiments, has no or low cross-reactivity with a microorganism cell wall or a component thereof. In some embodiments, the antibody has no or low cross-reactivity with lipopolysaccharide.

### EXAMPLES

Generally, the nomenclature used herein, and the laboratory procedures utilized in the present invention include chemical, molecular, biochemical, and cell biology techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); “Cell Biology: A Laboratory Handbook”, Volumes I-III Cellis, J. E., ed. (1994); The Organic Chemistry of Biological Pathways by John McMurry and Tadhg Begley (Roberts and Company, 2005); Organic Chemistry of Enzyme-Catalyzed Reactions by Richard Silverman (Academic Press, 2002); Organic Chemistry (6^{th} Edition) by Leroy “Skip” G Wade; Organic Chemistry by T. W. Graham Solomons and, Craig Fryhle.

### Material and Methods

### Fluorescence In-Situ Hybridization (FISH)

FISH probes were designed to a bacterial specific unique complementary 16S ribosomal RNA (rRNA) gene sequence, at the level of DNA. Fluorophore-labeled probe was ordered from Biomers (Germany). Up to 1×10⁹ of cultured bacteria or stool washed bacteria were fixed in 50% ice cold ethanol at −20° C. for 20 minutes. Following washing steps with hybridization buffer (0.9 M NaCl, 20 mM Tris pH 7.5, 0.01% SDS, 20% HiDi Formamide), bacteria were resuspended in 50 μl of hybridization buffer and probes (2 pmole/μl). Following 2 hours of incubation at 46° C. bacteria were washed with washing buffer (215 mM NaCl, 20 mM Tris pH 7.5, 5 mM EDTA) at 48° C. for 15 minutes 3 times.

### Antibody Production

For generating bacteria specific antibody, laying chickens were vaccinated subcutaneous with 50 μg of isolated bacteria, emulsified in Freund incomplete adjuvant (Sigma). Two weeks after a second boost injection, IgY antibodies were collected from the egg yolks, followed by sodium sulfate purification step. Antibody specificity was tested by flow cytometry.

### Flow Cytometry

Bacteria were washed with FACS buffer (PBS, 2% FCS, 1 mM EDTA, and 0.1% Sodium azide) and re-suspended in a concentration equivalent to 100 events/second (Fortessa low speed).

### Antibody Staining

Washed bacteria were incubated with the purified antibody for 30 minutes at 4° C., followed by a secondary goat anti-chicken IgY Alexa Fluor-647 conjugated.

### Magnetic Beads Capture

Magnetic beads (Thermo-Scientific) were coated with mouse anti-B. fragilis antibody. Beads were incubated with 1:1,000 Hoechst stained-bacteria for 30 minutes at 4° C. and analyzed by flow cytometry.

### Sorting Strategy

Bacteria are stained with 1:2,000 B. fragilis specific antibodies (Secondary Ab— 1:2,000 Rabbit (Fab2) anti Chicken IgY APC conjugate). Staining works at 4° C. and 25° C. in established staining buffer. Washing steps-1 ml staining buffer, centrifuge 6,500 g for 5 minutes. Optimal thresholds and voltage values were set on the AriaIII BD FACS. As control, the following groups were used: (1) Single stained bacteria; (2) Each bacteria with B. fragilis; and (3) Mix of all. In the mixtures, bacteria mixed, and then stained with specific Ab.

Staining and Isolating B. fragilis from Feces

B. fragilis were stained by metabolic click labeling— AF488 fluorophore. Fecal bacteria were washed and mixed with labeled B. fragilis and were detected by specific antibodies— APC fluorophore.

Briefly, bacteria were stained with 1:2,000 B. fragilis specific antibodies (Secondary Ab— 1:2,000 Rabbit (Fab2) anti Chicken IgY APC conjugate). Staining was performed at 25° C. in staining buffer (PBS, 10% FBS, 0.5 mM EDTA, pH 7.2). Washing steps-1 ml staining buffer, centrifuge 6,500 g for 5 minutes at 4° C. Optimal thresholds and voltage values were set for LSR Fortessa, BD analyzer.

Sorting B. fragilis from Feces

B. fragilis were stained for nucleic acids using Hoechst— AF405 Channel. Fecal bacteria were washed and mixed with Hoechst stained B. fragilis and were detected by specific antibodies— APC fluorophore.

Briefly, bacteria were stained with 1:2,000 B. fragilis specific antibodies (Secondary Ab— 1:2,000 Rabbit (Fab2) anti Chicken IgY Alexa Fluor 647 conjugate). Staining was performed at 25° C. in staining buffer (PBS, 10% FBS, 0.5 mM EDTA, pH 7.2). Washing steps-1 ml staining buffer, centrifuge 6,500 g for 5 minutes at 4° C. Optimal thresholds and voltage values were set for AriaIII, BD sorter. Bacteria were sorted to 4 tubes and were analyzed again by re-running the sorted bacteria.

### Example 1

Production of Specific Antibodies Raised Against FISH-Isolated Bacteria

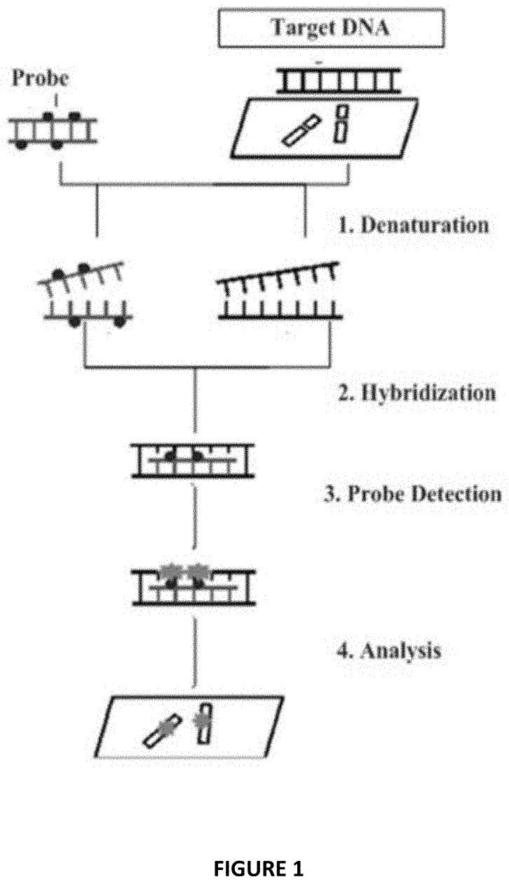

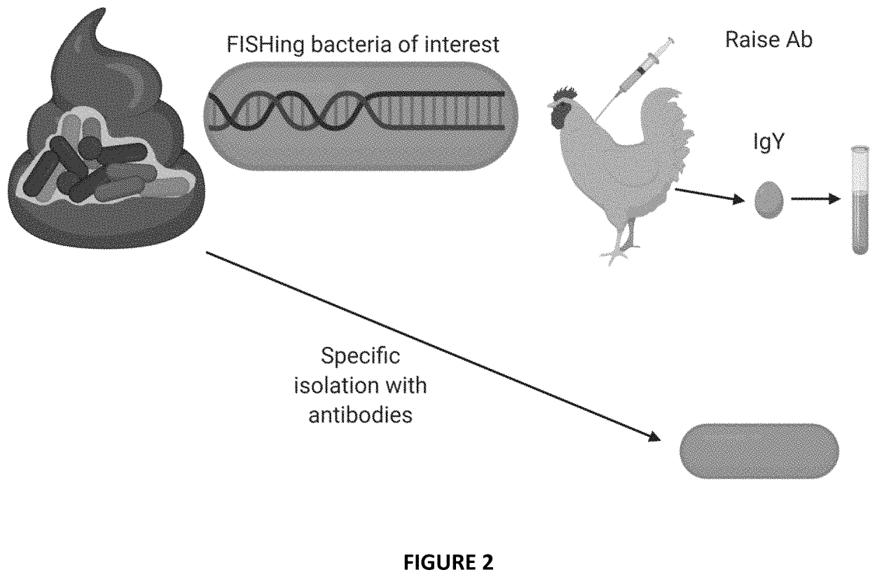

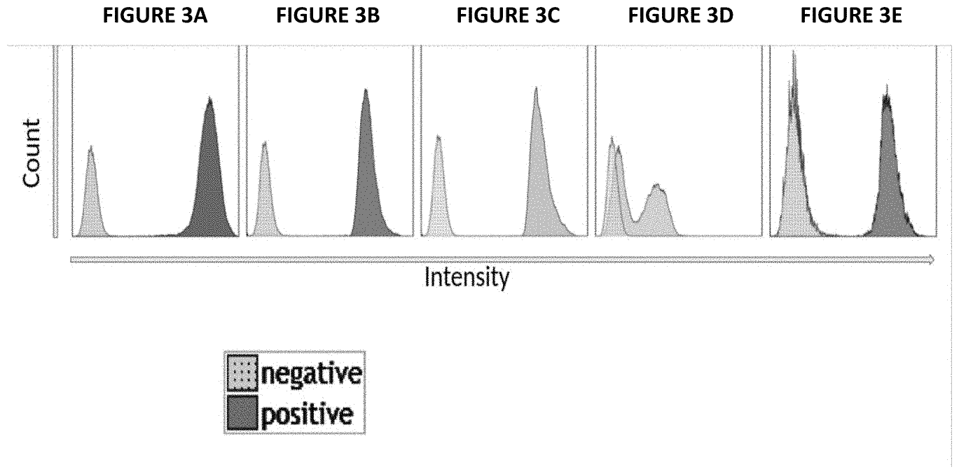

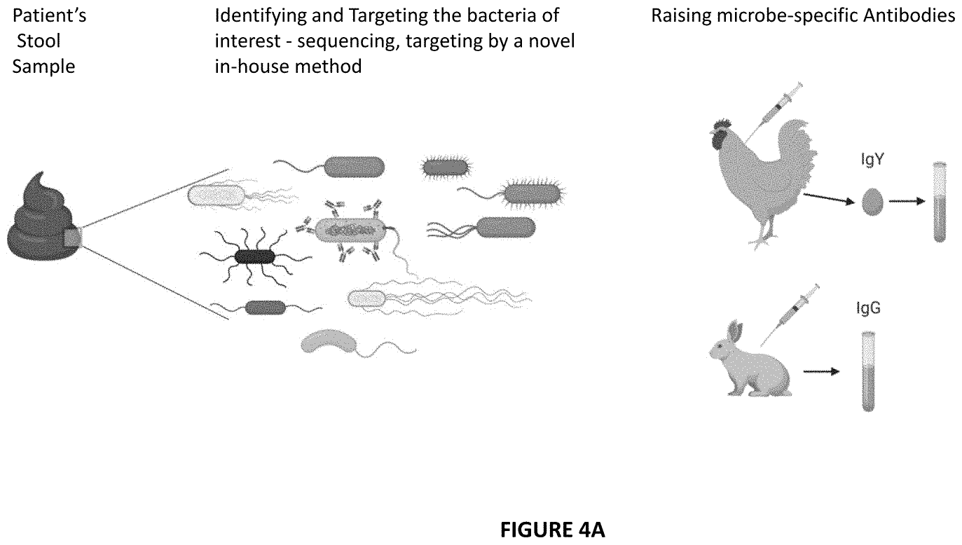

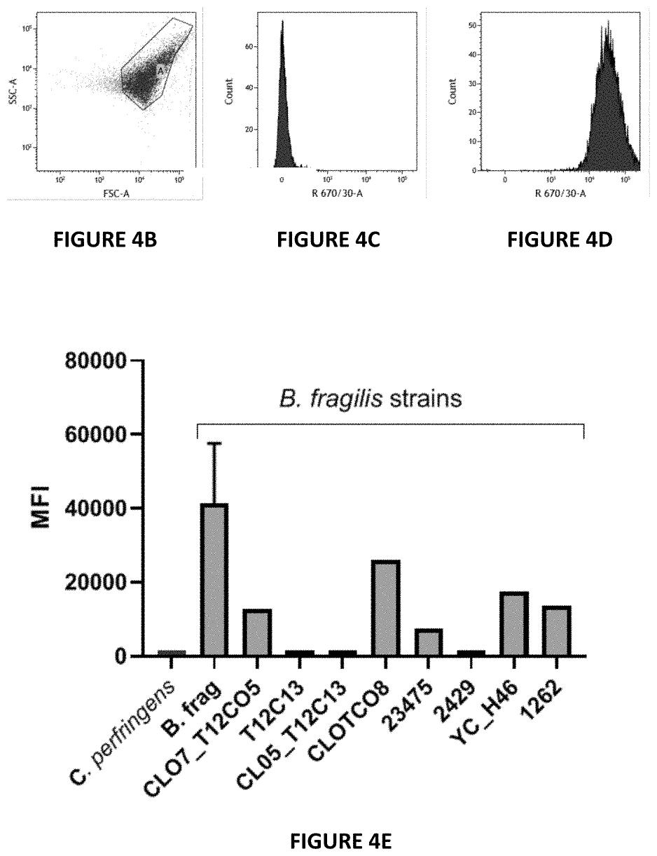

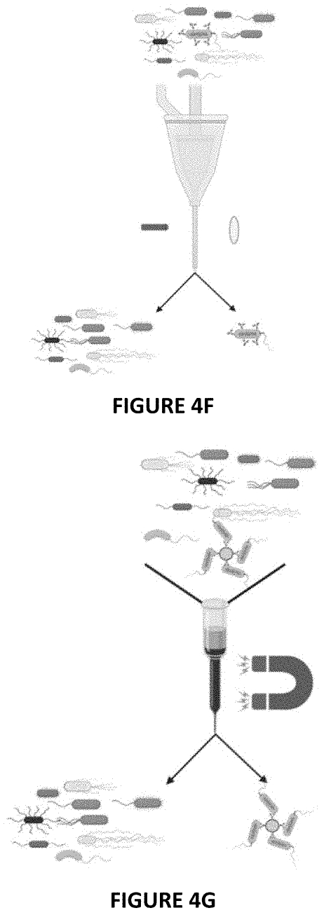

The inventors have shown that bacteria specific detection is feasible using complementary fluorescence labeled probes (FIG. 3) with minimal cross reactivity (FIGS. 3A-3E). The probe design was done using bioinformatics analysis for specific sequence unique to the bacterial 16S-rRNA gene (FIG. 1). Labeled bacteria were further isolated using fluorescence activated cell sorter (FACS). Following bacteria isolation, laying chickens or other animals are vaccinated with the isolated bacteria and specific antibodies are collected from the chicken's eggs or animal's serum (FIG. 2). To test the specificity of the raised antibodies, bacteria were incubated with the antibodies and tested by flow cytometry (FIGS. 4B-4D). The isolated antibodies were found to be specific to the vaccine bacterial strain with minimal cross reactivity between bacteria from other genus and with certain cross reactivity to other strains from the same bacterial species (FIG. 4E). Incubation of the bacteria with the specific antibody or with antibody coated magnetic beads, enabled the separation of the bacteria for the whole bacterial community by FACS (FIG. 4F) or by magnet (FIG. 4G), respectively.

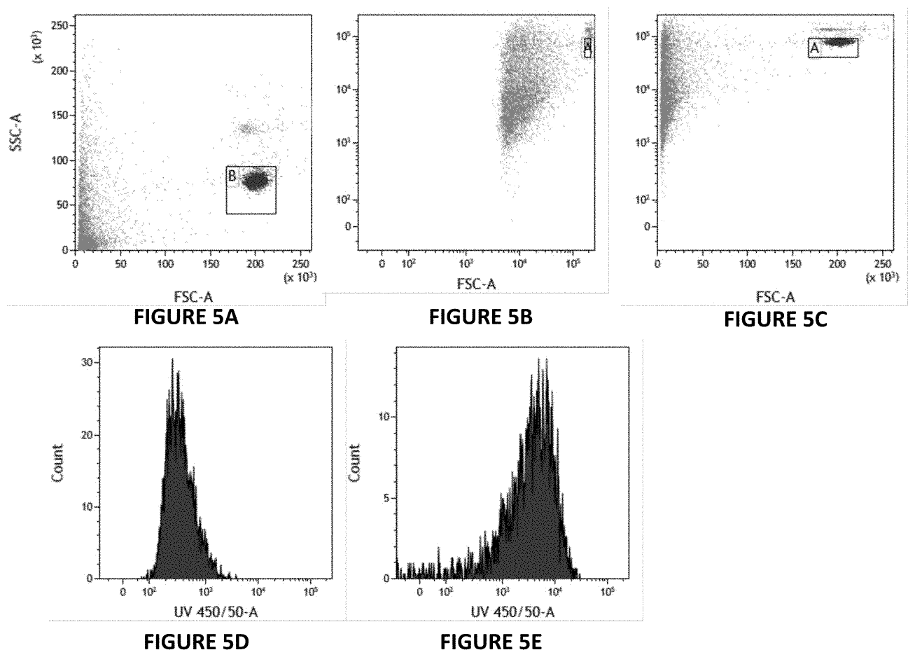

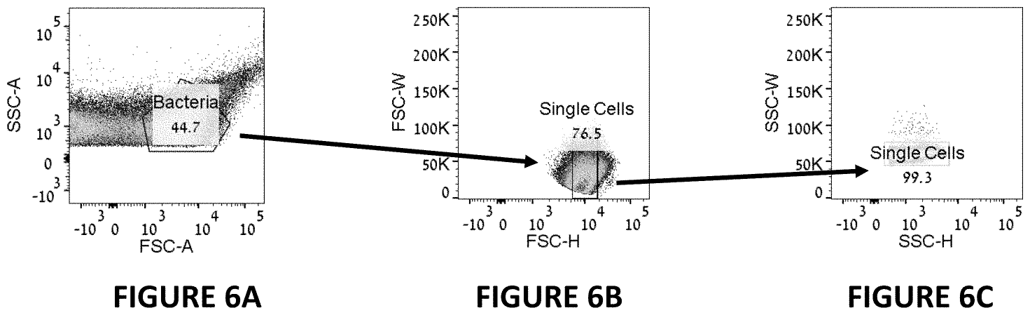

To verify the ability of the antibody-coated magnetic beads to specifically bind bacteria, beads were incubated with Hoechst stained B. fragilis and tested by flow cytometry (FIG. 5). Hoechst intensity of beads capturing B. fragilis (FIG. 5E) was compared to the background of non-relevant bacteria (FIG. 5D).

### Example 2

Detecting and Sorting Bacteroides fragilis in Feces

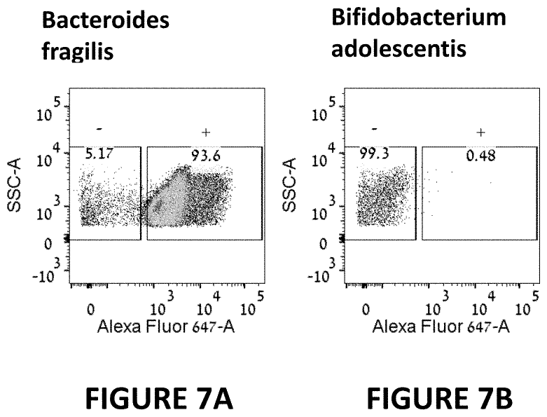

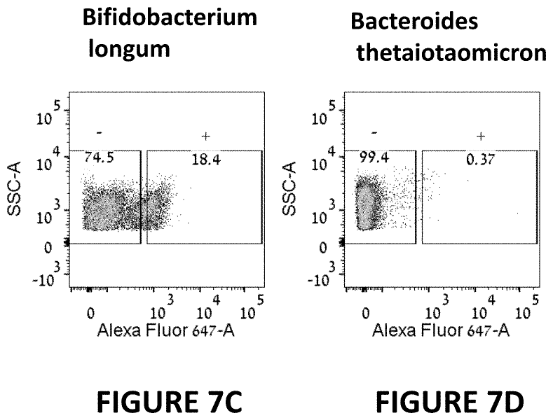

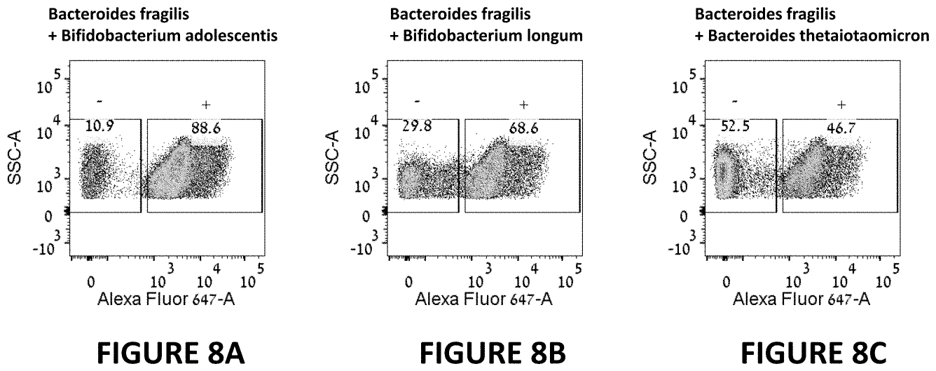

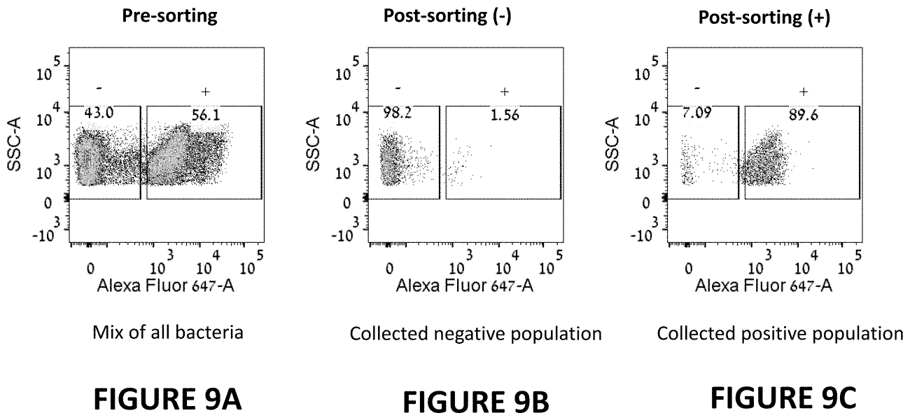

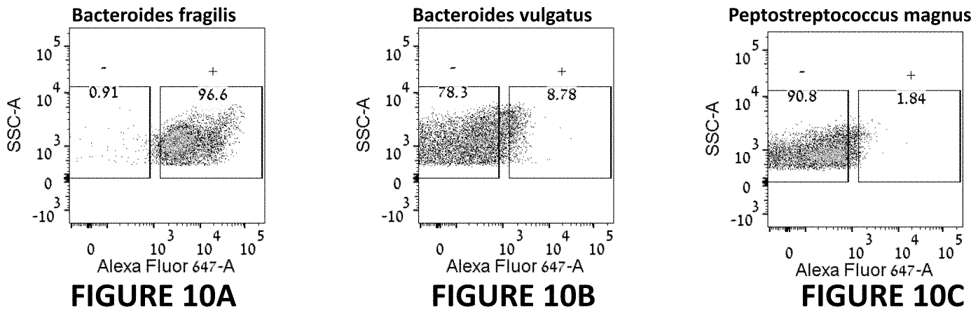

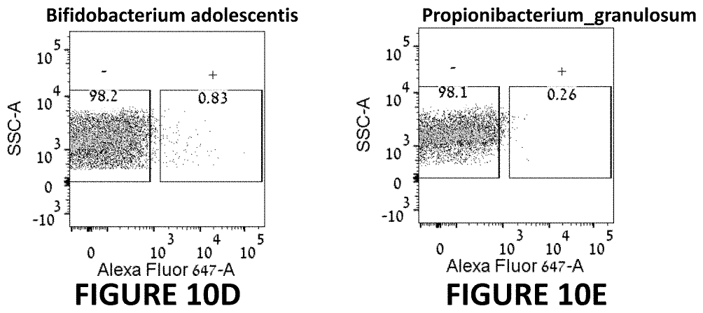

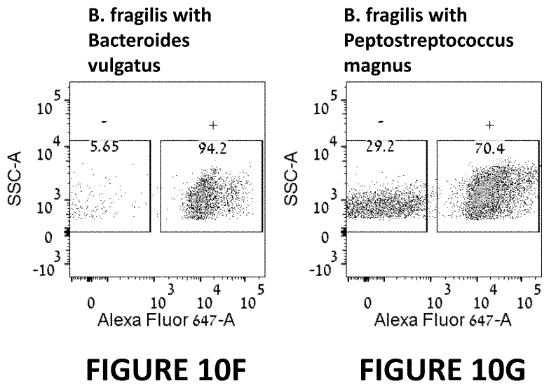

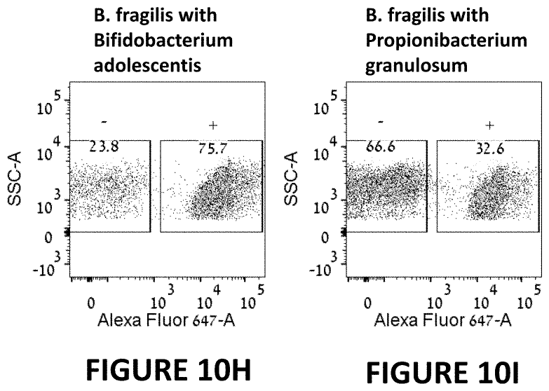

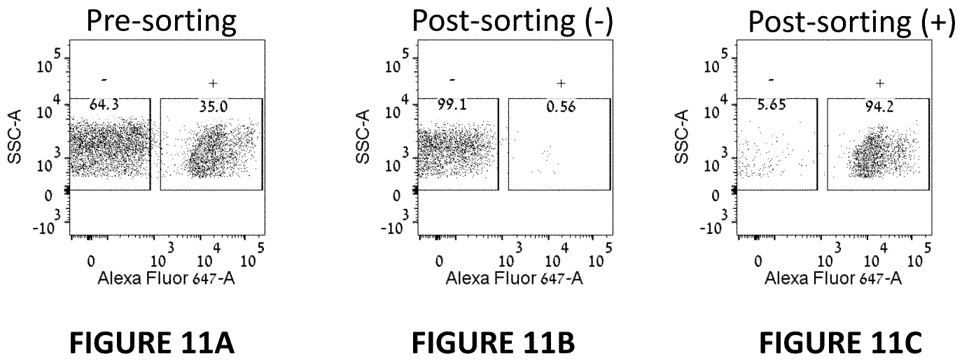

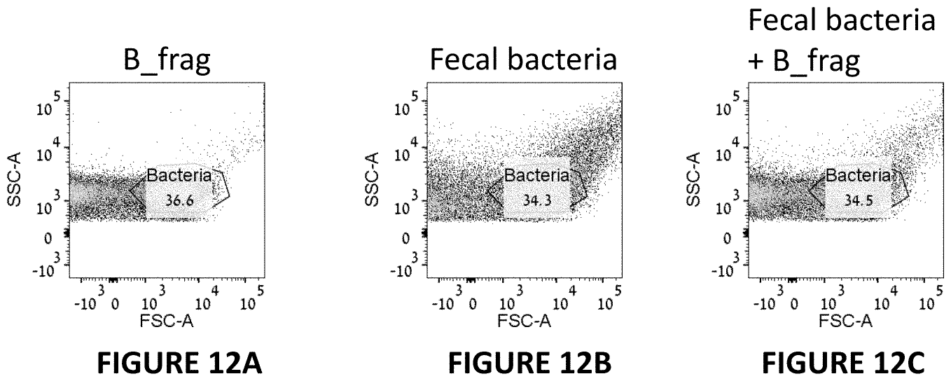

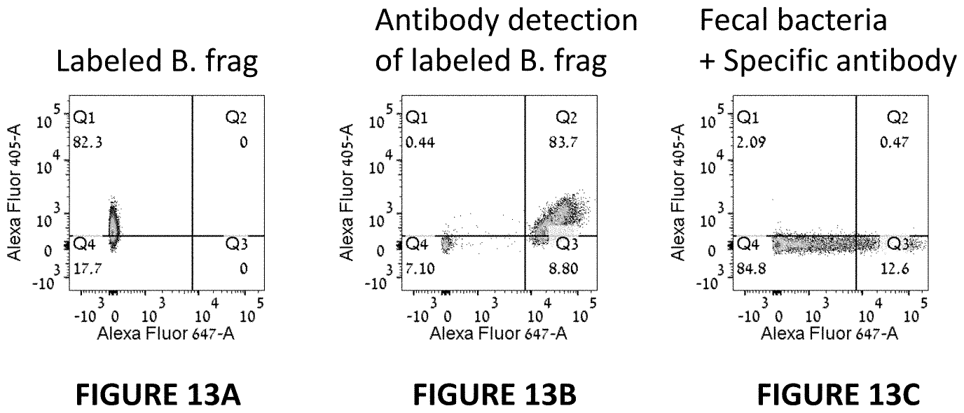

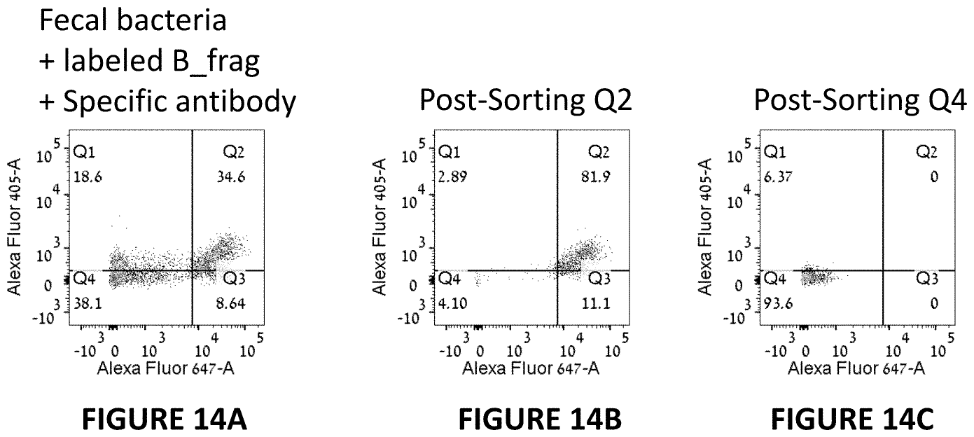

The inventors showed that cultured bacteria were recognizable with high specificity and efficiency (FIGS. 7, 8, and 11). IgY antibody, specifically targeting B. fragilis, showed low cross-reactivity to other cultured bacteria, yet some non-specific binding to unknown bacteria in the tested feces, was observed (FIG. 13C). Re-run of sorted bacteria from feces showed efficiency of 82% for stained B. fragilis (double positive) and 93% efficiency for negative (double negative—non-B. fragilis) bacteria (FIGS. 14B-14C).

While certain features of the invention have been described herein, many modifications, substitutions, changes, and equivalents will now occur to those of ordinary skill in the art. It is, therefore, to be understood that the appended claims are intended to cover all such modifications and changes as fall within the true spirit of the invention.

1. A method for isolating a target microorganism from a sample comprising a plurality of microorganisms, the method comprising contacting said sample with an antibody having specific affinity to said target microorganism, wherein said antibody being produced by immunizing a host organism using a selected target microorganism, wherein said selected target microorganism is selected by contacting a fraction of said sample with one or more polynucleotide molecules each having specific affinity to one target microorganism, and determining the presence of said target microorganism in said fraction of said sample,
thereby isolating a target microorganism from the sample comprising a plurality of microorganisms.

2. A method for isolating a target microorganism from a sample, the method comprising the steps of:
a. providing a fraction of said sample comprising a plurality of microorganisms;
b. contacting said fraction of said sample with one or more polynucleotide molecules each having specific affinity to one target microorganism, and determining the presence of said one or more target microorganism in said fraction of said sample;
c. selecting said one or more target microorganisms determined to be present in said fraction of said sample and immunizing a host organism using one of said selected target microorganisms, thereby producing an antibody having specific affinity to said one selected target microorganism; and
d. contacting said sample with said produced antibody having specific affinity to said one selected target microorganism,
thereby isolating a target microorganism from the sample.

3. The method of claim 1, wherein said microorganism is selected from the group consisting of: bacteria, fungi, archaea, protozoa, and algae.

4. The method of claim 1, wherein said selected microorganism is a specific species or a specific strain of a microorganism.

5. The method of claim 1, wherein said microorganism is a pathogenic microorganism.

6. The method of claim 1, wherein said microorganism is a probiotic microorganism.

7. The method of claim 1, wherein said contacting comprises contacting said sample with a plurality of polynucleotide molecules each having specific affinity to one target microorganism.

8. The method of claim 1, wherein said sample is selected from the group consisting of: a sample derived from a subject, a soil sample, and a water sample.

9. The method of claim 8, wherein said sample derived from a subject is a stool sample of said subject.

10. The method of claim 1, further comprising a step of enriching said sample with said isolated microorganism.

11. The method of claim 1, further comprising a step of depleting said isolated microorganism from said sample.

12. The method of claim 1, further comprising a step of determining said produced antibody has increased specific affinity to said one selected target microorganism compared to control.

13. A sample obtained by the method of claim 1.

14. A composition comprising the sample of claim 13 and a pharmaceutically acceptable carrier.

15. A method for treating a subject afflicted with a disease, the method comprising administering to said subject a therapeutically effective amount of the composition of claim 14.

16. The method of claim 15, wherein said disease is selected from the group consisting of: Clostridium difficile diarrhea, inflammatory bowel disease, irritable bowel disease, cancer, and diabetes.
